(19) United States
(12) Patent Application Publication
(10) Pub. No.: US 2006/0057161 A1
(43) Pub. Date: Mar. 16, 2006

(54) Detection of coronavirus infection

(76) Inventors: Chang-Jen Huang, Taipei (TW); Yueh-Chun Hsieh, Taipei (TW); Chih-Ming Chou, Taipei (TW); Ho-Sheng Wu, Taipei (TW); Ting-Hsiang Lin, Taipei (TW)

Correspondence Address: FISH & RICHARDSON PC, P.O. BOX 1022, MINNEAPOLIS, MN, 55440-1022, US

(21) Appl. No.: 10/942,698

(22) Filed: Sep. 16, 2004

(51) Int. Cl. C12Q 1/68; A61K 39/12

(52) U.S. Cl. 424/204.1; 435/6

(57) ABSTRACT

Isolated polypeptides containing one of SEQ ID NOs: 1-11. Also disclosed are (i) isolated nucleic acids encoding the polypeptides and related expression vectors and host cells; (ii) purified antibodies that recognize the polypeptides; and (iii) methods of producing the polypeptides, diagnosing infection with a coronavirus, and producing the antibodies.

### BACKGROUND

Coronavirus is a family of viruses that have the appearance of a corona when viewed under a microscope. Members of the coronavirus family cause hepatitis in mice, gastroenteritis in pigs, and respiratory infections in birds and humans. Among the more than 30 strains isolated so far, only three or four infect humans. For example, the severe acute respiratory syndrome (SARS), a newly found infectious disease, is associated with a novel coronavirus (Ksiazek et al., New England Journal Medicine, 2003, 348(20): 1953-1966). This life-threatening respiratory virus brought about worldwide outbreaks in 2003. There is a need for a method of diagnosing infection with SARS virus.

### SUMMARY

This invention relates to isolated polypeptides of SARS virus, which can be used in diagnosing infection with the virus. Listed below are the polypeptide and nucleotide sequences of SARS virus envelope (E), membrane (M), nucleocapsid (N), and spike (S) proteins.

SARS virus E protein
Polypeptide:

(SEQ ID NO: 1)

MYSFVSEETGTLIVNSVLLFLAFVVFLLVTTLAILTALRLCAYCCNIVNV
SLVKPTVYVYSRVKNLNSSEGVPDLLV
Nucleotide:

(SEQ ID NO: 12)

ATGTACTCATTCGTTTCGGAAGAAACAGGTACGTTAATAGTTAATAGCGT
ACTTCTTTTTCTTGCTTTCGTGGTATTCTTGCTAGTCACACTAGCCATCC
TTACTGCGCTTCGATTGTGTGCGTACTGCTGCAATATTGTTAACGTGAGT
TTAGTAAAACCAACGGTTTACGTCTACTCGCGTGTTAAAAATCTGAACTC
TTCTGAAGGAGTTCCTGATCTTCTGGTCTAA
SARS virus M protein
Polypeptide:

(SEQ ID NO: 2)

MADNGTITVEELKQLLEQWNLVIGFLFLAWIMLLQFAYSNRNRFLYIIKL
VFLWLLWPVTLACFVLAAVYRINWVTGGIAIAMACIVGLMWLSYFVASFR
LFARTRSMWSFNPETNILLNVPTGRGTIVTRPLMESELVIGAVIIRGHLR
MAGHPLGRCDIKDLPKEITVATSRTLSYYKLGASQRVGTDSGFAAYNRYR
IGNYKLNTDHAGSNDNIALLVQ
Nucleotide:

(SEQ ID NO: 13)

ATGGCAGACAACGGTACTATTACCGTTGAGGAGCTTAAACAACTCCTGGA
ACAATGGAACCTAGTAATAGGTTTCCTATTCCTAGCCTGGATTATGTTAC
TACAATTTGCCTATTCTAATCGGAACAGGTTTTTGTACATAATAAAGCTT
GTTTTCCTCTGGCTCTTGTGGCCAGTAACACTTGCTTGTTTTGTGCTTGC
TGCTGTCTACAGAATTAATTGGGTGACTGGCGGGATTGCGATTGCAATGG
CTTGTATTGTAGGCTTGATGTGGCTTAGCTACTTCGTTGCTTCCTTCAGG
CTGTTTGCTCGTACCCGCTCAATGTGGTCATTCAACCCAGAAACAAACAT
TCTTCTCAATGTGCCTCTCCGGGGGACAATTGTGACCAGACCGCTCATGG
AAAGTGAACTTGTCATTGGTGCTGTGATCATTCGTGGTCACTTGCGAATG
GCCGGACACCCCCTAGGGCGCTGTGACATTAAGGACCTGCCAAAAGAGAT
CACTGTGGCTACATCACGAACGCTTTCTTATTACAAATTAGGAGCGTCGC
AGCGTGTAGGCACTGATTCAGGTTTTGCTGCATACAACCGCTACCGTATT
GGAAACTATAAATTAAATACAGACCACGCCGGTAGCAACGACAATATTGC
TTTGCTAGTACAGTAA
SARS virus N protein
Polypeptide:

(SEQ ID NO: 3)

MSDNGPQSNQRSAPRITFGGPTDSTDNNQNGGRNGARPKQRRPQGLPNNT
ASWFTALTQHGKEELRFPRGQGVPINTNSGPDDQIGYYRRATRRVRGGDG
KMKELSPRWYFYYLGTGPEASLPYGANKEGIVWVATEGALNTPKDHIGTR
NPNNNAATVLQLPQGTTLPKGFYAEGSRGGSQASSRSSSRSRGNSRNSTP
GSSRGNSPARMASGGGETALALLLLDRLNQLESKVSGKGQQQQGQTVTKK
SAAEASKKPRQKRTATKQYNVTQAFGRRGPEQTQGNFGDQDLIRQGTDYK
HWPQIAQFAPSASAFFGMSRIGMEVTPSGTWLTYHGAIKLDDKDPQFKDN
VILLNKHIDAYKTFPPTEPKKDKKKKTDEAQPLPQRQKKQPTVTLLPAAD
MDDFSRQLQNSMSGASADSTQA
Nucleotide:

(SEQ ID NO: 14)

ATGTCTGATAATGGACCCCAATCAAACCAACGTAGTGCCCCCCGCATTAC
ATTTGGTGGACCCACAGATTCAACTGACAATAACCAGAATGGAGGACGCA
ATGGGGCAAGGCCAAAACAGCGCCGACCCCAAGGTTTACCCAATAATACT
GCGTCTTGGTTCACAGCTCTCACTCAGCATGGCAAGGAGGAACTTAGATT
CCCTCGAGGCCAGGGCGTTCCAATCAACACCAATAGTGGTCCAGATGACC
AAATTGGCTACTACCGAAGAGCTACCCGACGAGTTCGTGGTGGTGACGGC
AAAATGAAAGAGCTCAGCCCCAGATGGTACTTCTATTACCTAGGAACTGG
CCCAGAAGCTTCACTTCCCTACGGCGCTAACAAAGAAGGCATCGTATGGG
TTGCAACTGAGGGAGCCTTGAATACACCCAAAGACCACATTGGCACCCGC
AATCCTAATAACAATGCTGCCACCGTGCTACAACTTCCTCAAGGAACAAC
ATTGCCAAAAGGCTTCTACGCAGAGGGAAGCAGAGGCGGCAGTCAAGCCT
CTTCTCGCTCCTCATCACGTAGTCGCGGTAATTCAAGAAATTCAACTCCT
GGCAGCAGTAGGGGAAATTCTCCTGCTCGAATGGCTAGCGGAGGTGGTGA
AACTGCCCTCGCGCTATTGCTGCTAGACAGATTGAACCAGCTTGAGAGCA
AAGTTTCTGGTAAAGGCCAACAACAACAAGGCCAAACTGTCACTAAGAAA
TCTGCTGCTGAGGCATCTAAAAAGCCTCGCCAAAAACGTACTGCCACAAA
ACAGTACAACGTCACTCAAGCATTTGGGAGACGTGGTCCAGAACAAACCC
AAGGAAATTTCGGGGACCAAGACCTAATCAGACAAGGAACTGATTACAAA
CATTGGCCGCAAATTGCACAATTTGCTCCAAGTGCCTCTGCATTCTTTGG
AATGTCACGCATTGGCATGGAAGTCACACCTTCGGGAACATGGCTGACTT
ATCATGGAGCCATTAAATTGGATGACAAAGATCCACAATTCAAAGACAAC
GTCATACTGCTGAACAAGCACATTGACGCATACAAAACATTCCCACCAAC
AGAGCCTAAAAAGGACAAAAAGAAAAAGACTGATGAAGCTCAGCCTTTGC
CGCAGAGACAAAAGAAGCAGCCCACTGTGACTCTTCTTCCTGCGGCTGAC
ATGGATGATTTCTCCAGACAACTTCAAAATTCCATGAGTGGAGCTTCTGC
TGATTCAACTCAGGCATAA
SARS virus S protein
Polypeptide

(SEQ ID NO: 4)

MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELICDNPFFAVSKPMGTQTH
TMIFDNAFNCTFEYISDAFSLDVSEKSGNFKHLREFVFKNKLLGFLYVYK
GYQPIDVVRDLPSGFNTLKPIFKLPLGINITNFRAILTAFSPAQDIWGTS
AAAYFVGYLKPTTFMLKYDENGTITDAVDCSQNPLAELKCSVKSFEIDKG
IYQTSNFRVVPSGDVVRFPNITNLCPFGEVFNATKFPSVYAWERKKISNC
VADYSVLYNSTFFSTFKCYGVSATKLNDLCFSNVYADSFVVKGDDVRQIA
PGQTGVIADYNYKLPDDFMGCVLAWNTRNIDATSTGNYNYKYRYLRHGKL
RPFERDISNVPFSPDGKPCTPPALNCYWPLNDYGFYTTTGIGYQPYRVVV
LSFEILINAPATVCGPKLSTDLIKNQCVNFNFNGLTGTGVLTPSSKRFQP
FQQFGRDVSDFTDSVRDPKTSEILDISPCSFGGVSVITPGTNASSEVAVL
YQDXTNCTDVSTAIHADQLTPAWRIYSTGNNVFQTQAGCLIGAEHVDTSY
ECDIPIGAGICASYHTVSLLRSTSQKSIVAYTMSLGADSSIAYSNNTIAI
PTNFSISITTEVMPVSMAKTSVDCNNYICGDSTECANLLLQYGSFCTQLN
PALSGIAAEQDRNTREVFAQVKQMYKTPTLKYFGGFNFSQIIPDPIKPTK
RSFIEDLLFNKVTLIADAGFMKQYGECLGDINARDLICAQKFNGLTVLPP
LLTDDMIAAYTAALVSGTATAGWTFGAGAALQIPFAMQMAYRFNGIGVTQ
NVLYENQKQIANQFNKAISQIQESLTTTSTALGKLQDVTNQNAQALNTLV
KQLSSNFGAISSVLNDILSRLDKXTEAEVQIDRLITGRLQSLQTYVTQQL
IRAAEIRASANLAATKMSECVLGQSKRVDFCGKGYHLMSFPQAAPHGVVF
LHVTYVPSQERNFTTAPAICHEGKAYFPREGVFVFNGTSWFITQRNFFSP
QIITTDNTFVSGNCDVVIGIINNTVYDPLQPELDSFKEELDKYFKNHTSP
DVDLGDISGINASVVNIQKEIDRLNEVAKNLNESLIDLQELGKYEQYIKW
PWYVWLGFIAGLIAIVMVTILLCCMTSCCSCLKGACSCGSCCKFDEDDSE
PVLKGVKLHYT
Nucleotide:

(SEQ ID NO: 15)

ATGTTTATTTTCTTATTATTTCTTACTCTCACTAGTGGTAGTGACCTTGA
CCGGTGCACCACTTTTGATGATGTTCAAGCTCCTAATTACACTCAACATA
CTTCATCTATGAGGGGGGTTTACTATCCTGATGATATTTTTAGATCAGAC
ACTCTTTATTTAACTCAGGATTTATTTCTTCCATTTTATTCTAATGTTAC
AGGGTTTCATACTATTAATCATACGTTTGGCAACCCTGTCATACCTTTTA
AGGATGGTATTTATTTTGCTGCCACAGAGAAATCAAATGTTGTCCGTGGT
TGGGTTTTTGGTTCTACCATGAACAACAAGTCACAGTCGGTGATTATTAT
TAACAATTCTACTAATGTTGTTATACGAGCATGTAACTTTGAATTGTGTG
ACAACCCTTTCTTTGCTGTTTCTAAACCCATGGGTACACAGACACATACT
ATGATATTCGATAATGCATTTAATTGCACTTTCGAGTACATATCTGATGC
CTTTTCGCTTGATGTTTCAGAAAAGTCAGGTAATTTTAAACACTTACGAG
AGTTTGTGTTTAAAAATAAAGATGGGTTTCTCTATGTTTATAAGGGCTAT
CAACCTATAGATGTAGTTCGTGATCTACCTTCTGGTTTTAACACTTTGAA
ACCTATTTTTAAGTTGCCTCTTGGTATTAACATTACAAATTTTAGAGCCA
TTCTTACAGCCTTTTCACCTGCTCAAGACATTTGGGGCACGTCAGCTGCA
GCCTATTTTGTTGGCTATTTAAAGCCAACTACATTTATGCTCAAGTATGA
TGAAAATGGTACAATCACAGATGCTGTTGATTGTTCTCAAAATCCACTTG
CTGAACTCAAATGCTCTGTTAAGAGCTTTGAGATTGACAAAGGAATTTAC
CAGACCTCTAATTTCAGGGTTGTTCCCTCAGGAGATGTTGTGAGATTCCC
TAATATTACAAACTTGTGTCCTTTTGGAGAGGTTTTTAATGCTACTAAAT
TCCCTTCTGTCTATGCATGGGAGAGAAAAAAAATTTCTAATTGTGTTGCT
GATTACTCTGTGCTCTACAACTCAACATTTTTTTCAACCTTTAAGTGCTA
TGGCGTTTCTGCCACTAAGTTGAATGATCTTTGCTTCTCCAATGTCTATG
CAGATTCTTTTGTAGTCAAGGGAGATGATGTAAGACAAATAGCGCCAGGA
CAAACTGGTGTTATTGCTGATTATAATTATAAATTGCCAGATGATTTCAT
GGGTTGTGTCCTTGCTTGGAATACTAGGAACATTGATGCTACTTCAACTG
GTAATTATAATTATAAATATAGGTATCTTAGACATGGCAAGCTTAGGCCC
TTTGAGAGAGACATATCTAATGTGCCTTTCTCCCCTGATGGCAAACCTTG
CACCCCACCTGCTCTTAATTGTTATTGGCCATTAAATGATTATGGTTTTT
ACACCACTACTGGCATTGGCTACCAACCTTACAGAGTTGTAGTACTTTCT
TTTGAACTTTTAAATGCACCGGCCACGGTTTGTGGACCAAAATTATCCAC
TGACCTTATTAAGAACCAGTGTGTCAATTTTAATTTTAATGGACTCACTG
GTACTGGTGTGTTAACTCCTTCTTCAAAGAGATTTCAACCATTTCAACAA
TTTGGCCGTGATGTTTCTGATTTCACTGATTCCGTTCGAGATCCTAAAAC
ATCTGAAATATTAGACATTTCACCTTGCTCTTTTGGGGGTGTAAGTGTAA
TTACACCTGGAACAAATGCTTCATCTGAAGTTGCTGTTCTATATCAAGAT
GTTAACTGCACTGATGTTTCTACAGCAATTCATGCAGATCAACTCACACC
AGCTTGGCGCATATATTCTACTGGAAACAATGTATTCCAGACTCAAGCAG
GCTGTCTTATAGGAGCTGAGCATGTCGACACTTCTTATGAGTGCGACATT
CCTATTGGAGCTGGCATTTGTGCTAGTTACCATACAGTTTCTTTATTACG
TAGTACTAGCCAAAAATCTATTGTGGCTTATACTATGTCTTTAGGTGCTG
ATAGTTCAATTGCTTACTCTAATAACACCATTGCTATACCTACTAACTTT
TCAATTAGCATTACTACAGAAGTAATGCCTGTTTCTATGGCTAAAACCTC
CGTAGATTGTAATATGTACATCTGCGGAGATTCTACTGAATGTGCTAATT
TGCTTCTCCAATATGGTAGCTTTTGCACACAACTAAATCGTGCACTCTCA
GGTATTGCTGCTGAACAGGATCGCAACACACGTGAAGTGTTCGCTCAAGT
CAAACAAATGTACAAAACCCCAACTTTGAAATATTTTGGTGGTTTTAATT
TTTCACAAATATTACCTGACCCTCTAAAGCCAACTAAGAGGTCTTTTATT
GAGGACTTGCTCTTTAATAAGGTGACACTCGCTGATGCTGGCTTCATGAA
GCAATATGGCGAATGCCTAGGTGATATTAATGCTAGAGATCTCATTTGTG
CGCAGAAGTTCAATGGACTTACAGTGTTGCCACCTCTGCTCACTGATGAT
ATGATTGCTGCCTACACTGCTGCTCTAGTTAGTGGTACTGCCACTGCTGG
ATGGACATTTGGTGCTGGCGCTGCTCTTCAAATACCTTTTGCTATGCAAA
TGGCATATAGGTTCAATGGCATTGGAGTTACCCAAAATGTTCTCTATGAG
AACCAAAAACAAATCGCCAACCAATTTAACAAGGCGATTAGTCAAATTCA
AGAATCACTTACAACAACATCAACTGCATTGGGCAAGCTGCAAGACGTTG
TTAACCAGAATGCTCAAGCATTAAACACACTTGTTAAACAACTTAGCTCT
AATTTTGGTGCAATTTCAAGTGTGCTAAATGATATCCTTTCGCGACTTGA
TAAAGTCGAGGCGGAGGTACAAATTGACAGGTTAATTACAGGCAGACTTC
AAAGCCTTCAAACCTATGTAACACAACAACTAATCAGGGCTGCTGAAATC
AGGGCTTCTGCTAATCTTGCTGCTACTAAAATGTCTGAGTGTGTTCTTGG
ACAATCAAAAAGAGTTGACTTTTGTGGAAAGGGCTACCACCTTATGTCCT
TCCCACAAGCAGCCCCGCATGGTGTTGTCTTCCTACATGTCACGTATGTG
CCATCCCAGGAGAGGAACTTCACCACAGCGCCAGCAATTTGTCATGAAGG
CAAAGCATACTTCCCTCGTGAAGGTGTTTTTGTGTTTAATGGCACTTCTT
GGTTTATTACACAGAGGAACTTCTTTTCTCCACAAATAATTACTACAGAC
AATACATTTGTCTCAGGAAATTGTGATGTCGTTATTGGCATCATTAACAA
CACAGTTTATGATCCTCTGCAACCTGAGCTCGACTCATTCAAAGAAGAGC
TGGACAAGTACTTCAAAAATCATACATCACCAGATGTTGATCTTGGCGAC
ATTTCAGGCATTAACGCTTCTGTCGTCAACATTCAAAAAGAAATTGACCG
CCTCAATGAGGTCGCTAAAAATTTAAATGAATCACTCATTGACCTTCAAG
AATTGGGAAAATATGAGCAATATATTAAATGGCCTTGGTATGTTTGGCTC
GGCTTCATTGCTGGACTAATTGCCATCGTCATGGTTACAATCTTGCTTTG
TTGCATGACTAGTTGTTGCAGTTGCCTCAAGGGTGCATGCTCTTGTGGTT
CTTGCTGCAAGTTTGATGAGGATGACTCTGAGCCAGTTCTCAAGGGTGTC
AAATTACATTACACATAA

One aspect of the invention features an isolated polypeptide that contains SEQ ID NO: 1, 2, 3, or 4, or a fragment of SEQ ID NO: 4, such as amino acid (aa) 1-143 (“S1,” SEQ ID NO: 5), 144-262 (“S2,” SEQ ID NO: 6), 263-448 (“S3,” SEQ ID NO: 7), 449-690 (“S4,” SEQ ID NO: 8), 679-888 (“S5,” SEQ ID NO: 9), 884-1113 (“S6,” SEQ ID NO: 10), and 1032-1255 (“S7,” SEQ ID NO: 11). The isolated polypeptide is 76-2,000 amino acids, e.g., 76-1,500 amino acids, in length. In one embodiment, the polypeptide contains SEQ ID NO: 2, 3, 9, or 10.

An isolated polypeptide refers to a polypeptide substantially free from naturally associated molecules, i.e., it is at least 75% (i.e., any number between 75% and 100%, inclusive) pure by dry weight. Purity can be measured by any appropriate standard method, for example, by column chromatography, polyacrylamide gel electrophoresis, or HPLC analysis. An isolated polypeptide of the invention can be purified from a natural source, produced by recombinant DNA techniques, or by chemical methods.

The invention also features an isolated nucleic acid containing a sequence that encodes the above-mentioned polypeptide. Examples of the nucleic acid include SEQ ID NO: 12, 13, 14, and 15, as well as nucleotides 1-429, 430-786, 787-1344, 1345-2070, 2035-2664, 2650-3339, and 3094-3765 of SEQ ID NO: 15 (i.e., SEQ ID NOs: 16, 17, 18, 19, 20, 21, and 22, respectively). In one embodiment, the nucleic acid contains SEQ ID NO: 13, 14, 20, or 21.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different (i) DNA molecules, (ii) transfected cells, or (iii) cell clones, e.g., as these occur in a DNA library such as a cDNA or genomic DNA library. The nucleic acid described above can be used to express the polypeptide of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector of this invention includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vector can be introduced into host cells to produce the polypeptide of this invention. Also within the scope of this invention is a host cell that contains the above-described nucleic acid. Examples include E. coli cells, insect cells (e.g., using baculovirus expression vectors), yeast cells, plant cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and isolate the polypeptide from the cultured cell or the medium of the cell. Alternatively, the nucleic acid of this invention can be transcribed and translated in vitro, for example, using T7 promoter regulatory sequences and T7 polymerase.

One can use a polypeptide of this invention, e.g., a polypeptide containing SEQ ID NO: 2, 3, 9, or 10, to diagnose infection with a coronavirus, such as SARS-coronavirus, in a subject by determining presence of a specific antibody against the polypeptide in a first test sample (e.g., a serum sample) from the subject. Presence of the antibody (e.g., IgA, IgG, or IgM) in the test sample indicates the subject is infected with the coronavirus. One can further determine presence of a nucleotide sequence of the coronavirus in a second test sample, such as a swab sample, from the subject. The presence of the nucleotide sequence in the sample can be determined by PCR amplification with a pair of primers. Each of the primers can contain an oligo-nucleotide selected from the N, M, E or S gene region of the coronavirus and be 15-50 (e.g., 15-40) nucleotides in length. Exemplary pair of primers contain, respectively, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, or SEQ ID NOs: 41 and 42.

One can also use a polypeptide of this invention to produce antibodies in a subject that recognize a coronavirus, e.g., a SARS-coronavirus. To do so, one can administer to the subject with the polypeptide, or with an expression vector containing a nucleic acid encoding the polypeptide. Accordingly, within the scope of this invention is a composition containing the polypeptide (e.g., SEQ ID NO: 2, 3, 9, or 10) or an expression vector containing a nucleic acid encoding the polypeptide, and a pharmaceutical acceptable carrier.

Also within the scope of this invention is a purified antibody that recognizes and binds specifically to the polypeptide or its antigenic fragment. The antibody can be an IgA, IgG, or IgM. One can use the antibody to diagnose infection with a coronavirus in a subject determining presence of a polypeptide containing the sequence of SEQ ID NO: 1-11 in a test sample from the subject. Presence of the polypeptide in the test sample indicates the subject is infected with the coronavirus.

The details of one or more embodiments of the invention are set forth in the accompanying description below. Other advantages, features, and objects of the invention will be apparent from the detailed description and the claims.

### DETAILED DESCRIPTION

The present invention relates to polypeptides of the SARS virus. For example, within the scope of this invention is an isolated polypeptide containing one or more of SEQ ID NOs: 1-11. Since these polypeptides are antigenic and can induce immune response in a subject, they can be targeted for diagnosing and treating SARS.

A polypeptide of the invention can be obtained as a synthetic polypeptide or a recombinant polypeptide. To prepare a recombinant polypeptide, a nucleic acid encoding it can be linked to another nucleic acid encoding a fusion partner, e.g., Glutathione-S-Transferase (GST), 6x-His epitope tag, or M 13 Gene 3 protein. A vector containing the nucleic acid can be introduced into suitable host cells via conventional transformation or transfection techniques, such as calcium phosphate or calcium chloride co-precipitation, DEAE-dextran-mediated transfection, lipofection, or electroporation. After being transformed or transfected, the host cells can be cultured in a medium to express the fusion protein. The protein can then be isolated from the host cells or from the culture medium using standard techniques. It can be further treated, e.g., by enzymatic digestion, to remove the fusion partner and obtain the recombinant polypeptide of this invention.

If an expressed polypeptide is fused to one of the tags described above, the polypeptide can be easily purified from a clarified cell lysate or culture medium with an appropriate affinity column, e.g., Ni²⁺ NTA resin for hexa-histidine, glutathione agarose for GST, amylose resin for maltose binding protein, chitin resin for chitin binding domain, and antibody affinity columns for epitope tagged proteins. The polypeptide can be eluted from the affinity column, or if appropriate, cleaved from the column with a site-specific protease. If the polypeptide is not tagged for purification, routine methods in the art can be used to develop procedures to isolate it from cell lysates or the media. See, e.g., Scopes, RK (1994) Protein Purification: Principles and Practice, 3rd ed., New York: Springer-Verlag.

As mention above, a polypeptide of this invention can be targeted for diagnosing SARS. More specifically, the presence of antibodies against the polypeptide in a subject indicates that the subject is infected with SRAS-Cov. Thus, one can determine the presence or absence of the antibodies in a test sample from the subject by detecting a binding between the antibodies and the polypeptide, thereby diagnosing SARS. Examples of techniques for detecting antibody-polypeptide binding include ELISAs, immunoprecipitations, immunofluorescence, EIA, RIA, and Western blotting analysis. The amino acid composition of a polypeptide of the invention may vary without disrupting the ability of the polypeptide to bind to its specific antibody. For example, it can contain one or more conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in SEQ ID NO: 1 is preferably replaced with another amino acid residue from the same side chain family. Alternatively, mutations can be introduced randomly along all or part of SEQ ID NO: 1, such as by saturation mutagenesis, and the resultant mutants can be screened for the antibody-binding ability.

A polypeptide of this invention can also be targeted for treating SARS in a subject. Accordingly, also within the scope of this invention is an immunogneic or antigenic composition that contains a pharmaceutically acceptable carrier and an effective amount of a polypeptide or nucleotide of the invention. The composition can be used to produce antibodies in a subject that recognize a coronavirus, e.g., a SARS-coronavirus. The presence of the antibodies in the subject can protect the subject from an infection with the coronavirus. The carriers used in the composition are selected on the basis of the mode and route of administration, and standard pharmaceutical practice. Suitable pharmaceutical carriers and diluents, as well as pharmaceutical necessities for their use, are described in Remington's Pharmaceutical Sciences. An adjuvant, e.g., a cholera toxin, Escherichia coli heat-labile enterotoxin (LT), liposome, or immune-stimulating complex (ISCOM), can also be included in the composition, if necessary.

The amount of composition administered will depend, for example, on the particular peptide antigen in the polypeptide, whether an adjuvant is co-administered with the antigen, the type of adjuvant co-administered, the mode and frequency of administration, and the desired effect (e.g., protection or treatment), as can be determined by one skilled in the art. In general, the polypeptide is administered in amounts ranging between 1 μg and 100 mg per adult human dose. If adjuvants are co-administered, amounts ranging between 1 ng and 1 mg per adult human dose can generally be used. Administration is repeated as necessary, as can be determined by one skilled in the art. For example, a priming dose can be followed by three booster doses at weekly intervals. A booster shot can be given at 8 to 12 weeks after the first administration, and a second booster can be given at 16 to 20 weeks, using the same formulation. Sera can be taken from the individual for testing the immune response elicited by the composition against the polypeptide. Methods of assaying antibodies against a specific antigen are well known in the art. Additional boosters can be given as needed. By varying the amount of polypeptide and frequency of administration, the protocol can be optimized for eliciting a maximal production of the antibodies.

A polypeptide of the invention can be used to generate antibodies in animals (for production of antibodies) or humans (for treatment of diseases). Methods of making monoclonal and polyclonal antibodies and fragments thereof in animals are known in the art. See, for example, Harlow and Lane, (1988) Antibodies: A Laboratory Manual, Cold Spring Harbor Laboratory, New York. The term “antibody” includes intact molecules as well as fragments thereof, such as Fab, F(ab′)₂, Fv, scFv (single chain antibody), and dAb (domain antibody; Ward, et. al. (1989) Nature, 341, 544). These antibodies can be used for detecting the polypeptide, e.g., in determining whether a test sample from a subject contains SARS virus. These antibodies are also useful for treating SARS since they interfere with cell-binding and entry of the virus.

In general, a polypeptide of the invention can be coupled to a carrier protein, such as KLH, mixed with an adjuvant, and injected into a host animal. Antibodies produced in that animal can then be purified by peptide affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum.

Polyclonal antibodies, heterogeneous populations of antibody molecules, are present in the sera of the immunized subjects. Monoclonal antibodies, homogeneous populations of antibodies to a polypeptide of this invention, can be prepared using standard hybridoma technology (see, for example, Kohler et al. (1975) Nature 256, 495; Kohler et al. (1976) Eur. J. Immunol. 6, 511; Kohler et al. (1976) Eur. J. Immunol. 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sic. USA 80, 2026, and the EBV-hybridoma technique (Cole et al. (1983) Monoclonal Antibodies and Cancer Therapy, Alan R. Less, Inc., pp. 77-96). Such antibodies can be of any immunoglobulin class including Gig, IBM, IgA, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production.

In addition, techniques developed for the production of “chimeric antibodies” can be used. See, e.g., Morrison et al. (1984) Proc. Natl. Acad. Sic. USA 81, 6851; Neutered et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage library of single chain Fv antibodies. Single chain antibodies are formed by linking the heavy and light chain fragments of the Fv region via an amino acid bridge. Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments. Antibodies can also be humanized by methods known in the art. For example, monoclonal antibodies with a desired binding specificity can be commercially humanized (Scotgene, Scotland; and Oxford Molecular, Palo Alto, Calif.). Fully human antibodies, such as those expressed in transgenic animals are also features of the invention (see, e.g., Green et al. (1994) Nature Genetics 7, 13; and U.S. Pat. Nos. 5,545,806 and 5,569,825).

The above-described antibodies can also be used for diagnosing or treating SARS. Also within the scope of this invention is a method of treating SARS, e.g., by administering to a subject in need thereof an effective amount of an antibody. Subjects to be treated can be identified as having, or being at risk for acquiring, a condition characterized by SARS. This method can be performed alone or in conjunction with other drugs or therapy. The term “treating” is defined as administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result, e.g., as described above, in a treated subject. In one in vivo approach, a therapeutic composition (e.g., a composition containing an antibody) is administered to a subject. Generally, the antibody is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily. The dosage required depends on the choice of the route of administration; the nature of the formulation; the nature of the subject's illness; the subject's size, weight, surface area, age, and sex; and other drugs being administered. The efficacy of the pharmaceutical composition can be preliminarily evaluated in vitro. For in vivo studies, the composition can be injected into an animal (e.g., the transgenic mouse model described in Blumberg H et al., Cell 104:9, 2001) and its effects on SARS are then accessed.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

Recombinant SARS-CoV proteins were expressed and purified. RT-PCR was used to obtain the regions encoding the SARS-CoV proteins, N, M, E, and fragments of S (S1, S2, S3, S4, S5, S6, and S7) from RNA extracted from the Urbani strain of SARS-CoV (GenBank accession number AY278741). The genome of this strain was 29,727 nucleotides in length and kindly provided by Centers for Disease Control and Prevention, USA (CDC-US). The sequences of the primer pairs used in the PCR were listed in Table 1 below. Each amplicon had Bam HI/Sal I or Bam HI/Hind III restriction sites at its two ends. The sizes for all amplicons were also listed in Table 1.

TABLE 1
Primers for amplifying DNA fragments of SARS-CoV

| | | | | | |
|---|---|---|---|---|---|
| | SEQ ID | | | Restriction | Amplicon |
| Gene | NO.: | Primers | Sequences | sites | size (bp) |

| | | | | | |
|---|---|---|---|---|---|
| N | 23 | SA-NF | 5′-CTGGATCCATGTCTGATAATGGACCCCAT-3′ | BamHI | 1269 |
| | 24 | SA-NR | 5′-GCGTCGACTTATGCCTGAGTTGAATCAGC-3′ | Sal I | |
| M | 25 | SA-MF | 5′-CTGGATCCATGGCAGACAACGGTAGT-3′ | BamHI | 666 |
| | 26 | SA-MR | 5′-GCGTCGACCTGTACTAGCAAAGCAAT-3′ | Sal I | |
| E | 27 | SA-EF | 5′-CTGGATCCATGTACTCATTCGTTTCGGAA-3′ | BamHI | 231 |
| | 28 | SA-ER | 5′-GGAAGCTTTTAGACCAGAAGATCAGGAAC-3′ | HindIII | |
| S1 | 29 | SA-SF1 | 5′-CTGGATCCATGTTTATTTTCTTATTATTT-3′ | BamHI | 429 |
| | 30 | SA-SR1 | 5′-GCAAGCTTGGGTTTAGAAACAGCAAAGAA-3′ | HindIII | |
| S2 | 31 | SA-SF2 | 5′-CTGGATCCATGGGTACACAGACACAT-3 | BamHI | 353 |
| | 32 | SA-SR2 | 5′-GCAAGCTTGTAGTGGCTTTAAATAG-3′ | HIndIII | |
| S3 | 33 | SA-SF3 | 5′-CTGGATCCATGCTCAAGTATGATGAA-3′ | BamHI | 560 |
| | 34 | SA-SR3 | 5′-CTAAGCTTGCCATGTCTAAGATACCT-3′HIndIII | | |
| S4 | 35 | SA-SF4 | 5′-CTGGATCCATGAGGCCCTTTGAGAGA-3′ | BamHI | 725 |
| | 36 | SA-SR4 | 5-GCAAGCTTGAGTAAGCAATTGAACTA-3′ | HIndIII | |
| S5 | 37 | SA-SF5 | 5′-CTGGATCCATGTCTTTAGGTGCTGAT-3′ | BamHI | 630 |
| | 38 | SA-SR5 | 5′-GCAAGCTTGAACCTATATGCCATTTG-3′ | HindIII | |
| S6 | 39 | SA-SF6 | 5′-CTGGATCCATGGCATATAGGTTCAAT-3′ | BamHI | 690 |
| | 40 | SA-SR6 | 5′-GGAAGCTTGCCAATAACGACATCACA-3′ | HindIII | |
| S7 | 41 | SA-SF7 | 5′-CTGGATCCATGTCCTTCCCACAAGCA-3′ | BamHI | 663 |
| | 42 | SA-5R7 | 5′-GCAAGCTTTTATGTGTAATGTAATTTGAGACC-3′ | HindIII | |

The amplified products were purified and cloned into the pQE30 expression vector (Qiagen, GmbH, Germany) by standard techniques. The resulting vectors were transformed into E. coli JM109 cells (Invitrogen, Carlsbad, Calif.) and verified by DNA sequencing on an ABI 3730 DNA Analyzer (Applied Biosystems, Foster City, Calif.). The verified expression vectors were transformed into E. coli JM109. Colonies of the transformed E. coli cells were inoculated into LB broths respectively in the presence of ampicillin at 100 μg/ml, and cultured overnight at 37° C. until the optical density at 600 nm (OD600 nm) of the culture reached 1.2. To induce the expression of the recombinant proteins, isopropyl-β-D-thiogalactopyranoside (IPTG) was added to each culture to a final concentration of 1.0 mM. After the culture was grown for 4 hours, all the recombinant proteins accumulated in the bacteria as inclusion bodies. The cells were then harvested by centrifugation, and the proteins were purified. Briefly, 5 ml of a culture was resuspended in 1 ml of phosphate buffer saline (PBS), pH 7. The cells in it were disrupted by sonication in ice bath at a 10 second interval for 3 times. After centrifugation at 13,000 rpm for 5 minutes, the pellet was resuspended in an eppendorf vial containing 1.5% sarcosine, 10 mM Tris-HCl buffer (pH 7.0), and vertexed at room temperature for 1 hour until the lysate became clear. The resuspension was centrifuged at 13,000 rpm for 5 minutes. The supernatant was collected and mixed with BD TALON™ metal affinity resins (BD Biosciences, BD Biosciences, San Jose, Calif.). The resultant mixture was incubated at 4° C. overnight with slight agitation. Then, the resins were collected by centrifugation and washed twice with 10 mM Tris-HCl-1 M NaCl. Proteins bound to the resins were eluted with gradient imidazole solution according to the manufacturer's instructions to eliminate bacterial contaminants. The proteins were then run on 12% SDS-PAGE and then, either stained with Coomassie Blue dye or transferred to a polyvinylidene difluoride membrane (PVDF Immobilon P, pore size 0.45 μm, Millipore, USA) for blotting.

It was found that after induction with IPTG, most of the proteins were synthesized and present in inclusion bodies. As expected, SDS-PAGE analysis showed that bacterially expressed SARS-CoV N, E, S2, S5, and S6 proteins had molecular weights of 46, 10, 14, 23, and 25 kDa, respectively. However, the apparent molecular weight of recombinant M protein was 35 kDa, which is larger than the calculated size (approximately 25 kDa), possibly due to the high content of hydrophobic amino acid residues (49%, 109/221).

### EXAMPLE 2

Western blot analysis was conducted for detecting antibodies to SARS-CoV. The above-described recombinant proteins were pooled and tested, by Western blot analysis, for their ability of binding to antibodies in serum samples of SARS patients.

According to WHO criteria, a suspected case was classified as a person who, after Nov. 1, 2002, (i) had a high fever (>38° C.), cough or breathing difficulty and (ii) resided in or traveled to an area with recent local transmission of SARS during the 10 days prior to onset of symptoms. A suspect case was classified as a probable case if his or her X-ray radiographic evidence of infiltrates was consistent with pneumonia or respiratory distress syndrome.

From hospitalized patients in northern part of Taiwan, 54 patients (18 males and 38 females) were determined to be probable SARS cases. Their serum samples were collected from the 2^{nd} through 41^{st} day after the onset of illness. More specifically, 36 paired-serum samples (collected at both the acute stage, i.e., day 1 to day 12 after illness onset and the convalescent stage of the illness) and 18 single-serum samples (collected at either the acute stage or the convalescent stage of the illness, i.e., day 19 to day 41 after illness onset) were obtained from, respectively, at the acute stage or at the convalescent stage of the illness. All of the serum samples were examined for SARS-CoV by RT-PCR. It was found that 48 were positive and 42 were negative. The primers used for RT-PCR were synthesized according to CDC-US recommendation. The handling of specimens, including collection, aliquot or dilution of specimens, and nucleic acid extraction or RT-PCR assay, was conducted in biosafety level 2 (BSL-2) laboratories.

The above-described serum samples were then analyzed by Western blot. More specifically, equal amounts of purified recombinant proteins were mixed, subjected to SDS-PAGE, and transferred to PVDF membranes. The membranes were blocked with 5% skim milk in PBS for 2 hours at room temperature and then were cut into strips (0.5 cm×8 cm). The protein loadings in all strips were theoretically equal. Each of the serum samples was 1:500 diluted with 5% skim milk. Two milliliters of each diluted serum was incubated with each strip overnight at 4° C. On the following day, the strips were washed with PBS-0.2% Tween-20 for 3 times (10 min each) and incubated with 2 ml of 1:1000 diluted goat anti-human IgG, IgA, or IgM conjugated with horseradish peroxidase (Savyon, Ashdod, Israel) at room temperature for 2 hours. After washing in PBS-0.2% Tween-20 as described above, the strips were incubated with an ECL solution (PerkinElmer Life Sciences, Boston, Mass.) for 1 minute. The strips were then dried and exposed to x-ray films to visualize the reaction. Two sera from healthy people were used as negative controls.

It was found that the N protein was recognized by IgA, IgG, or IgM. The S(S5 or S6) and M proteins were recognized by IgA or IgG, but hardly by IgM. Two proteins, E and S2, were not recognized by IgA, IgG, or IgM in any of the serum samples tested.

These results indicate that recombinant N, M, S5, and S6 proteins could be used as diagnostic markers for SARS-CoV infection.

As mentioned above, among all serum samples examined, 48 were found to be SARS-CoV positive by RT-PCR. Table 2 below summarizes the Western blot results from these 48 samples

TABLE 2
Antibody responses to different viral antigens in 48 positive samples

| | IgA | Positive | IgG | | IgA or IgG | |
|---|---|---|---|---|---|---|
| Viral Antigens | No. of Pos. | Rate % | No. of Pos. | % | No. of Pos. | % |
| M | 10 | 20.8% | 6 | 12.5% | | |
| N + M | 3 | 6.3% | 5 | 10.4% | | |
| N + M + S6 | 3 | 6.3% | 1 | 2.1% | | |
| N + S5 | 4 | 8.3% | 4 | 8.3% | | |
| N + S6 | 3 | 6.3% | 2 | 4.2% | | |
| N + S5 + S6 | 3 | 6.3% | 0 | 0.0% | | |
| N-related^{a} | 26 | 54.2% | 18 | 37.5% | 27 | 56.3% |
| M | 0 | 0.0% | 1 | 2.1% | 1 | 2.1% |
| S5 | 2 | 4.2% | 3 | 6.3% | | |
| S6 | 3 | 6.3% | 0 | 0.0% | | |
| S5 + S6 | 2 | 4.2% | 1 | 2.1% | | |
| S^{b} | 7 | 14.6% | 4 | 8.3% | 7 | 14.6% |
| N-related^{a} + M + S^{b} | 33 | 68.8% | 23 | 47.9% | 35 | 72.9% |
| Total specimens | 48 | | | | | |

^{a}N-related represents the total number of N, N + M, N + M + S6, N + S5, N + S6, N + S5 + S6.
^{b}S represents the total number of S5, S6, and S5 + S6

As shown in Table 2, the blotting using N-related antigens (N, N+M, N+M+S, and N+S) had a positive rate of 54.2% (26 of 48) for detecting IgA, and 37.5% (19 of 48) for IgG. If the results of IgA and IgG were combined, the positive rate was increased to 56.3% (27 of 48). When using S antigens (S5, S6, and S5+S6), 14.6% (7 of 48) of the patients were determined as positive for IgA, and 8.3% (4 of 48) for IgG. The positive rate was not raised even if both IgA and IgG were detected. Taken together, when using pooled antigens (N, M, S5 and S6), the positive rate was of 68.8% (33/48) for IgA, 47.9% (23/48) for IgG, and 72.9% (35/48) for IgA or IgG.

### EXAMPLE 3

The levels of immunoglobulins to the above-described recombinant proteins were profiled to elucidate a patient's immune response to various antigens of SARS-CoV. Line diagrams were created based on the Western blot results described above by Sigma Plot version 8.0. The levels of immunoglobulins were normalized against those of the two health people.

Sensitivity [true positive/(true positive+false negative)] and specificity [true negative/(true negative+false positive)] were calculated as described in Büttner J. Clin. Chem. Clin. Biochem. 15:1-12; 2003. The antibody response of different immunoglobin classes to SARS-CoV recombinant proteins was plotted according to the optical density of Western blots, which was scanned and quantified using the TotalLab software (Nonlinear Dynamics, NC). The value of each band was normalized with the control serum. The results were subjected to Sigma Plot for curve plotting and pair to pair t-test. For all statistical analyses, P<0.05 was considered statistically significant.

It was found that N protein possessed the major antigenicity of inducing IgA, IgG, and IgM. Noticeably, anti-N protein IgA appeared at as early as day 2 or day 3 after illness onset, and increased by 7-fold within the first 3-4 days and progressively increased by 15-fold within one month. The increase in anti-N protein IgA was significantly higher than that in anti-M protein and anti-S protein by paired t-test (P<0.01, and P<0.05, respectively). The behavior of anti-N protein IgM or IgG was similar to that of anti-N IgA, except that they appeared on day 10 and day 16, respectively, after the onset of illness. The M protein could be detected by IgA or IgG. The level of antibodies against M protein was much lower than that of anti-N protein. Interestingly, a few patients had IgA antibodies against the spike protein at the early stage (day 2 to day 3), but most of other patients had the similar response at the convalescent stage (16 to day 21 after illness onset).

### EXAMPLE 4

Data obtained from the above-described Western blot assay was compared with the results from whole virus-based immunofluorescence assay (IFA) to evaluate the sensitivity and specificity of the Western blot assay.

More specifically, Vero E6 cells were grown in MEM containing 10% fetal bovine serum at 35° C. In a BSL-3 laboratory, the cells (at a density of 80%) were infected with SARS-CoV (10⁶/ml). The virus culturing and viral antigens preparation were also conducted in a BSL-3 laboratory. After cytopathic effects (CPE) appeared, the cells were treated with 0.025% trypsin and spotted on multi-well slides. The slides were dried in a closed heating container and were fixed in acetone for 15 minutes. Afterwards, all experiments were carried out in a BSL-2 laboratory. 10 μl of diluted serum sample (starting from 1:100) was placed into each well of the slide, and incubated at 37° C. for 30 minutes. After washing twice with PBS for 5 minutes each, 10 μl of 1:100 diluted specific anti-human gamma globulins labeled with FITC (Zymed Laboratories, South San Francisco, Calif.) was added to each well, and incubated at 37° C. for 30 minutes. After washing twice with PBS, the slides were observed under a fluorescence microscope. The results are summarized in Table 3 below.

TABLE 3
Comparison of results obtained from Western blot assay and IFA

| Western blot | Sensitivity^{a} | Specificity^{b} | Overall agreement^{c} |
|---|---|---|---|
| IgA or IgG | 89.1% (41/46) | 88.6% (39/44) | 88.9% (80/90) |
| IgA | 73.9% (34/46) | 97.7% (43/44) | 85.6% (77/90) |
| IgG | 91.3% (42/46) | 88.6% (39/44) | 90.0% (81/90) |

^{a}Number of true positives divided by total number of IFA-positive sera.
^{b}Number of true negatives divided by total number of IFA-negative sera.
^{c}Sum of the number of true positives and true negatives divided by total serum samples.

As shown in Table 3, the results obtained by the Western blot-based method correlate well with those obtained by IFA. These indicate that the Western blot-based method is quite sensitive and specific.

### EXAMPLE 4

The above-described Western blot-based method was compared with RT-PCR-based method. Briefly, samples from 54 probable SARS cases were examined by Western blot and RT-PCR. Throat swab specimens were used for RT-PCR. For Western blot, paired serum samples and single serum samples were obtained from 36 and 18 patients, respectively. The results obtained by both methods are summarized in Table 4 below:

TABLE 4
Comparison of Western blot-based method and RT-PCR-based method

| | Stage of | | | | | | RT-PCR (+) or | |
|---|---|---|---|---|---|---|---|---|
| Serum type | serum collected | Case No. | RT-PCR (+) | | Western blot (+) | | Western blot (+) | |
| Single serum | Acute stage | 8 | 50% | (4/8) | 25% | (2/8) | 50% | (4/8) |
| | convalescent stage | 10 | 40% | (4/10) | 60% | (6/10) | 70% | (7/10) |
| Paired sera | Acute stage/convalescent stage | 36 | 50% | (18/36) | 75% | (27/36) | 77.8% | (28/36) |
| Total | | 54 | 48.1% | (26/54) | 64.8% | (35/54) | 72.2% | (39/54) |

As shown in Table 4, in the single serum group, the RT-PCR-based method and Western blot-based method were 50% and 25% accurate, respectively, if specimens collected at the acute stage were used. They are 40% and 60% accurate, respectively, if specimens collected at the convalescent stage were used. In the paired sera group, the RT-PCR-based method and Western blot-based method are 50% and 75% accurate, respectively. Overall, the positive rates are 48.1% for the RT-PCR-based method, and 64.8% for the Western blot-based method. With combination of both methods, the accuracy went up to 72.2% (39/54). The results suggest that the RT-PCR-based method was more sensitive than the Western blot-based method at the acute phase. In contrast, the Western blot-based method was more sensitive at the convalescent phase. The recombination of both methods increased the accuracy.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. An isolated polypeptide comprising the sequence of SEQ ID NO: 1, 2, 3, 4, 5, 6, 7, 8, 9, 10, or 11.

2. The polypeptide of claim 1, wherein the polypeptide is 76-2,000 amino acids in length.

3. The polypeptide of claim 2, wherein the polypeptide is 76-1,500 amino acids in length.

4. The polypeptide of claim 1, wherein the polypeptide contains SEQ ID NO: 2, 3, 9, or 10.

5. An isolated nucleic acid comprising a sequence that encodes the polypeptide of claim 1.

6. The nucleic acid of claim 5, wherein the sequence is SEQ ID NO: 12, 13, 14, 15, 16, 17, 18, 19, 20, 21, or 22.

7. The nucleic acid of claim 6, wherein the sequence is SEQ ID NO: 13, 14, 20, or 21.

8. A vector comprising the nucleic acid of claim 5.

9. A host cell comprising the nucleic acid of claim 5.

10. The host cell of claim 9, wherein the host cell is an E. coli, yeast, insect, plant, or mammalian cell.

11. The host cell of claim 10, wherein the host cell is an E. coli cell.

12. A method of producing a polypeptide, the method comprising
culturing the host cell of claim 9 in a medium under conditions permitting expression of the polypeptide, and
isolating the polypeptide.

13. A purified antibody that binds specifically to the polypeptide of claim 1 or a fragment thereof.

14. The antibody of claim 13, wherein the antibody is an IgA, IgG, or IgM.

15. A method of diagnosing infection with a coronavirus in a subject, comprising:
providing a first test sample from a subject, and
determining presence of a specific antibody against a polypeptide of claim 1 in the first test sample,
wherein presence of the antibody indicates the subject is infected with the coronavirus.

16. The method of claim 15, wherein the antibody is an IgA, IgG, or IgM.

17. The method of claim 15, wherein the first test sample is a serum sample.

18. The method of claim 15, wherein the coronavirus is a SARS-coronavirus.

19. The method of claim 15, further comprising
providing a second test sample from the subject, and
determining presence of a nucleotide sequence of the coronavirus in the second test sample.

20. The method of claim 19, wherein the second test sample is a swab sample.

21. The method of claim 19, wherein the presence of the nucleotide sequence is determined by PCR amplification with a pair of primers, each primer containing an oligo-nucleotide selected from the N, M, E or S gene region of the coronavirus and being 15-50 nucleotides in length.

22. The method of claim 21, wherein each primer is 15-40 nucleotides in length.

23. The method of claim 22, wherein the pair of primers contain, respectively, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, or SEQ ID NOs: 41 and 42.

24. The method of claim 15, wherein the polypeptide contains SEQ ID NO: 2, 3, 9, or 10.

25. The method of claim 24, wherein the first test sample is a serum sample.

26. The method of claim 24, wherein the coronavirus is a SARS-coronavirus.

27. The method of claim 24, further comprising
providing a second test sample from the subject, and
determining presence of a nucleotide sequence of the coronavirus in the second test sample.

28. The method of claim 24, wherein the antibody is an IgA, IgG, or IgM.

29. The method of claim 28, wherein the first test sample is a serum sample.

30. The method of claim 29, wherein the coronavirus is a SARS-coronavirus.

31. The method of claim 30, further comprising
providing a second test sample from the subject, and
determining presence of a nucleotide sequence of the coronavirus in the second test sample.

32. The method of claim 31, wherein the presence of the nucleotide sequence is determined by PCR amplification with a pair of primers, each primer containing an oligo-nucleotide selected from the N, M, E or S gene region of the coronavirus, wherein each primer is 15-50 nucleotides in length.

33. The method of claim 32, wherein each primer is 15-40 nucleotides in length.

34. The method of claim 33, wherein the pair of primers contain, respectively, SEQ ID NOs: 23 and 24, SEQ ID NOs: 25 and 26, SEQ ID NOs: 27 and 28, SEQ ID NOs: 29 and 30, SEQ ID NOs: 31 and 32, SEQ ID NOs: 33 and 34, SEQ ID NOs: 35 and 36, SEQ ID NOs: 37 and 38, SEQ ID NOs: 39 and 40, or SEQ ID NOs: 41 and 42.

35. The method of claim 34, wherein the second test sample is a swab sample.

36. A composition comprising
a polypeptide of claim 1 or an expression vector containing a nucleic acid encoding the polypeptide; and
a pharmaceutical acceptable carrier.

37. The composition of claim 36, wherein the polypeptide contains SEQ ID NO: 2, 3, 9, or 10.

38. A method of producing antibodies which recognize coronavirus in a subject, the method comprising administering to the subject a polypeptide of claim 1, or an expression vector containing a nucleic acid encoding the polypeptide.

39. The method of claim 38, wherein the polypeptide contains SEQ ID NO: 2, 3, 9, or 10.

40. The method of claim 39, wherein the coronavirus is a SARS-coronavirus.


SEQUENCE LISTING (42 sequences)

SEQ ID NO: 1 - protein, 76 aa, SARS coronavirus
MYSFVSEETGTLIVNSVLLFLAFVVFLLVTLAILTALRLCAYCCNIVNVSLVKPTVYVYSRVKNLNSSEGVPDLLV

SEQ ID NO: 2 - protein, 221 aa, SARS coronavirus
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQ

SEQ ID NO: 3 - protein, 422 aa, SARS coronavirus
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 4 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 5 - protein, 143 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKP

SEQ ID NO: 6 - protein, 119 aa, SARS coronavirus
MGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTF

SEQ ID NO: 7 - protein, 186 aa, SARS coronavirus
MLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKL

SEQ ID NO: 8 - protein, 242 aa, SARS coronavirus
RPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYS

SEQ ID NO: 9 - protein, 210 aa, SARS coronavirus
MSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRF

SEQ ID NO: 10 - protein, 230 aa, SARS coronavirus
MAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIG

SEQ ID NO: 11 - protein, 224 aa, SARS coronavirus
MSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 12 - DNA, 231 bp, SARS coronavirus
atgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaa

SEQ ID NO: 13 - DNA, 666 bp, SARS coronavirus
atggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaa

SEQ ID NO: 14 - DNA, 1269 bp, SARS coronavirus
atgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataa

SEQ ID NO: 15 - DNA, 3768 bp, SARS coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 16 - DNA, 429 bp, SARS coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaaccc

SEQ ID NO: 17 - DNA, 357 bp, SARS coronavirus
atgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacattt

SEQ ID NO: 18 - DNA, 558 bp, SARS coronavirus
atgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagctt

SEQ ID NO: 19 - DNA, 726 bp, SARS coronavirus
aggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactct

SEQ ID NO: 20 - DNA, 630 bp, SARS coronavirus
atgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttc

SEQ ID NO: 21 - DNA, 690 bp, SARS coronavirus
atggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggc

SEQ ID NO: 22 - DNA, 672 bp, SARS coronavirus
atgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacaca

SEQ ID NO: 23 - DNA, 29 bp, Artificial Sequence
  Primer
ctggatccatgtctgataatggaccccat

SEQ ID NO: 24 - DNA, 29 bp, Artificial Sequence
  Primer
gcgtcgacttatgcctgagttgaatcagc

SEQ ID NO: 25 - DNA, 26 bp, Artificial Sequence
  Primer
ctggatccatggcagacaacggtact

SEQ ID NO: 26 - DNA, 26 bp, Artificial Sequence
  Primer
gcgtcgacctgtactagcaaagcaat

SEQ ID NO: 27 - DNA, 29 bp, Artificial Sequence
  Primer
ctggatccatgtactcattcgtttcggaa

SEQ ID NO: 28 - DNA, 29 bp, Artificial Sequence
  Primer
gcaagcttttagaccagaagatcaggaac

SEQ ID NO: 29 - DNA, 29 bp, Artificial Sequence
  Primer
ctggatccatgtttattttcttattattt

SEQ ID NO: 30 - DNA, 29 bp, Artificial Sequence
  Primer
gcaagcttgggtttagaaacagcaaagaa

SEQ ID NO: 31 - DNA, 26 bp, Artificial Sequence
  Primer
ctggatccatgggtacacagacacat

SEQ ID NO: 32 - DNA, 26 bp, Artificial Sequence
  Primer
gcaagcttgtagttggctttaaatag

SEQ ID NO: 33 - DNA, 26 bp, Artificial Sequence
  Primer
ctggatccatgctcaagtatgatgaa

SEQ ID NO: 34 - DNA, 26 bp, Artificial Sequence
  Primer
ctaagcttgccatgtctaagatacct

SEQ ID NO: 35 - DNA, 26 bp, Artificial Sequence
  Primer
ctggatccatgaggccctttgagaga

SEQ ID NO: 36 - DNA, 26 bp, Artificial Sequence
  Primer
gcaagcttgagtaagcaattgaacta

SEQ ID NO: 37 - DNA, 26 bp, Artificial Sequence
  Primer
ctggatccatgtctttaggtgctgat

SEQ ID NO: 38 - DNA, 26 bp, Artificial Sequence
  Primer
gcaagcttgaacctatatgccatttg

SEQ ID NO: 39 - DNA, 26 bp, Artificial Sequence
  Primer
ctggatccatggcatataggttcaat

SEQ ID NO: 40 - DNA, 26 bp, Artificial Sequence
  Primer
gcaagcttgccaataacgacatcaca

SEQ ID NO: 41 - DNA, 26 bp, Artificial Sequence
  Primer
ctggatccatgtccttcccacaagca

SEQ ID NO: 42 - DNA, 32 bp, Artificial Sequence
  Primer
gcaagcttttatgtgtaatgtaatttgacacc